(19) United States
(12) Patent Application Publication
(10) Pub. No.: US 2001/0006656 A1
(43) Pub. Date: Jul. 5, 2001

(54) METHODS AND COMPOSITIONS FOR INHIBITING INFLAMMATION ASSOCIATED WITH PULMONARY DISEASE

(75) Inventors: JOHN M. HARLAN, SEATTLE, WA (US); ROBERT K. WINN, BAINBRIDGE ISLAND, WA (US); LI LIU, REDMOND, WA (US)

Correspondence Address: CAMPBELL & FLORES LLP, 4370 LA JOLLA VILLAGE DRIVE, 7TH FLOOR, SAN DIEGO, CA, 92122, US

(73) Assignee: UNIVERSITY OF WASHINGTON

(21) Appl. No.: 09/251,707

(22) Filed: Feb. 17, 1999

(51) Int. Cl. A61K 9/00

(52) U.S. Cl. 424/400

(57) ABSTRACT

The present invention provides an aerosol formulation of a 3-hydroxy-3-methyl-glutaryl coenzyme A (HMG-CoA) reductase inhibitor. The HMG-CoA reductase inhibitor can be, for example, a statin such as lovastatin, pravastatin, simvastatin, cerivastatin, fluvastatin, atorvastatin or mevastatin. The invention also provides a method of treating a pulmonary disease with an aerosol formulation of a HMG-CoA reductase inhibitor.

This invention was made with government support under grant number HL 18645, HL 07312 and HL 30542 awarded by the United States Public Health Service. The government has certain rights in the invention.

### BACKGROUND OF THE INVENTION

The present invention relates generally to the fields of medicine and molecular pathology and, more specifically, to methods of treating an inflammatory lung disease.

### BACKGROUND INFORMATION

A variety of pulmonary diseases are associated with inflammation, including acute and chronic diseases. Pulmonary diseases associated with inflammation include, for example, asthma, interstitial pneumonitis, emphysema, chronic bronchitis, adult respiratory distress syndrome (ARDS) and cystic fibrosis.

Many of the lung diseases associated with inflammation have a significant impact on human health, quality of life and productivity. For example, approximately 5% of the United States population has signs or symptoms of asthma. Chronic obstructive pulmonary disease, including chronic bronchitis and emphysema, is the fourth leading cause of death in the United States. In addition, the United States has approximately 100,000 cases of adult respiratory distress syndrome (ARDS), which can follow systemic or pulmonary insults. Cystic fibrosis is the most common lethal genetic disease in Caucasians, affecting approximately one in 2,000 births among Americans of European descent. Therefore, inflammatory lung diseases have a major impact on human health.

The early stages of an inflammatory response involve the release of signaling molecules, called chemotactic factors, that recruit inflammatory cells to the site of inflammation. Inflammatory cells such as neutrophils are capable of releasing enzymes,, oxygen radicals and cytokines that can result in significant tissue damage. In lung diseases associated with inflammation, inflammatory cells migrate into the lung, where the damage caused by the infiltrating inflammatory cells results in respiratory distress associated with the diseases.

Some lung diseases associated with inflammation can be treated, for example, with anti-inflammatory agents such as corticosteroids. However, corticosteroids have disadvantages. For example, corticosteiroids can cause complete immunosuppression and can induce “wasting” syndrome, diabetes, hypertension, peptic ulcer, osteoporosis, fatty liver, cataracts and other undesirable side effects.

There exists a need for safe and effective anti-inflammatory agents that reduce the severity of lung diseases associated with inflammation. The present invention satisfies this need and provides related advantages as well.

### SUMMARY OF THE INVENTION

The present invention provides an aerosol formulation of a 3-hydroxy-3-methyl-glutaryl coenzyme A (HMG-CoA) reductase inhibitor. The HMG-CoA reductase inhibitor can be, for example, a statin such as lovastatin, pravastatin, simvastatin, cerivastatin, fluvastatin, atorvastatin or mevastatin. The invention also provides a method of treating a pulmonary disease with an aerosol formulation of a HMG-CoA reductase inhibitor.

### BRIEF DESCRIPTION OF THE DRAWINGS

FIG. 1 shows the effect of lovastatin on cell adhesion. A. Jurkat cells were treated with the indicated concentration of lovastatin. Cell adhesion to fibronectin was induced with either phorbol 12-myristate 13-acetate (PMA) (100 ng/ml) or the β1-integrin-activating monoclonal antibody 8A2 (2 μg/ml). Percent cell adherence is shown. B. U937 cells were treated with the indicated concentration of lovastatin. Cell adhesion to laminin was induced with either PMA (100 ng/ml) monoclonal antibody 8A2 (2 μg/ml). Percent cell adherence is shown.

FIG. 2 shows that the MEK-1 specific inhibitor, PD-98059, does not inhibit PMA-stimulated Jurkat cell adherence to fibronectin. Jurkat cells were treated with the indicated concentration of PD-98059, and cell adherence was induced with PMA. Percent cell adhesion is shown.

FIG. 3 shows that the effect of lovastatin on PMA-stimulated adhesion is due to geranylgeranylation. A. Jurkat cells were co-incubated with 5 μM all trans-geranylgeranol (GGOH) and the indicated concentration of lovastatin. Cell adhesion was induced with PMA. Percent cell adherence is shown. B. Jurkat cells were treated with 5 μM lovastatin and the indicated concentration of GGOH. Cell adhesion was induced with PMA or 8A2, and percent cell adherence is shown. C. Jurkat cells were co-incubated with all trans-farnesol (FOH) (5 μM) and the indicated concentration of lovastatin. Cell adhesion was induced with PMA, and percent cell adherence is shown.

FIG. 4 shows the effect of intra nasal (i.n.) administration of lovastatin on LPS-stimulated cell migration into lung. A. Mice were stimulated by i.n. administration of lipopolysaccharide (LPS) in the presence of the indicated concentrations of lovastatin administered i.n. At 24 and 48 hours, lung lavage fluids were obtained, and cell counts of neutrophils (PMN) were determined. The “*” indicates that differences were statistically significant when compared with no lovastatin (control) group (p<0.05). B. Mice were treated as in FIG. 1A, and the cell counts of mononuclear cells were determined. Differences between groups were not statistically significant (p<0.05).

FIG. 5 shows the effect of intra peritoneal (i.p.) administration of lovastatin on LPS-stimulated cell migration into lung. Neutrophil (PMN) cell counts in lung lavage fluids were determined at 24 and 48 hours after i.n. instillation of LPS and i.p. administration of lovastatin. The lovastatin group was statistically different from the control (no lovastatin) group (P<0.05).

FIG. 6 shows the effect of i.n. administration of lovastatin on PMA-stimulated cell migration into lung. Neutrophil (PMN) and mononuclear cell counts were determined in lung lavage fluids following i.n. instillation of PMA together with lovastatin. The lovastatin group was statistically different from the no lovastatin (control) group (p<0.05).

FIG. 7 shows the effect of lovastatin administration on the level of cytokine KC in lung lavage fluids. A. The concentration of the cytokine KC in lung lavage fluids was obtained 24 and 48 hours after i.n. instillation of LPS together with i.n. lovastatin. The “*” indicates that differences were statistically significant when compared to no lovastatin (control) group (p<0.05). B. The concentration of the cytokine KC in lung lavage fluids was determined 24 hours after i.n. instillation of LPS and i.p. administration of lovastatin. The difference in KC concentration was statistically significant when the lovastatin and control groups were compared (p<0.05).

FIG. 8 shows the correlation between KC concentration and the number of neutrophils (PMN) in lung lavage fluids following i.n. instillation of LPS with lovastatin administered either i.n. (A) or i.p. (B). The data are represented as a linear regression best-fit for each of the groups. Correlation between PMNs and KC was significant in both cases (p<0.05).

FIG. 9 shows the effect of lovastatin on tumor necrosis factor-α (TNF) in lavage fluids. A. The concentration of TNF in lavage fluids was determined at 24 and 48 hours after i.n. instillation of LPS together with i.n. lovastatin (“i.n. 24 hr” and “i.n. 48 hr.” respectively). B. The concentration of TNF in lavage fluids was determined at 24 hours after i.n. instillation of LPS and i.p. administration of lovastatin. The “*” indicates that differences were statistically significant when compared with no lovastatin (control) group (p<0.05).

FIG. 10 shows airway responsiveness in ovalbumin sensitized mice treated with lovastatin. Mice were sensitized by administration of ovalbumin and challenged with methacholine. The effect of lovastatin on airway responsiveness was determined. Airway responsiveness was measured as enhanced phase (Penh).

FIG. 11 shows the effect of lovastatin on leukocyte infiltration in lung lavage fluids of ovalbumin sensitized mice. Mice were sensitized by administration of ovalbumin and challenged with methacholine. The effect of lovastatin on the infiltration of leukocytes into lung lavage fluids was determined. Differential cell counts were determined for white blood cells (WBC), eosinophils (Eos), neutrophils (PMN) and mononuclear cells (Mono).

FIG. 12 shows the change in mouse hind limb diameter following ischemia and reperfusion. Mouse hind leg was subjected to ischemia and reperfusion, and lovastatin (7 μg/g) or carrier (control) was administered i.p. at the beginning of ischemia. The increase in leg diameter was measured over a 24 hour period.

FIG. 13 shows the chemical structure of selected HMG-CoA reductase inhibitors.

### DETAILED DESCRIPTION OF THE INVENTION

The present invention relates to the use of 3-hydroxy-3-methyl-glutaryl coenzyme A (HMG-CoA) reductase inhibitors as anti-inflammatory agents for the treatment of inflammatory lung disease. In addition to their role in regulating cholesterol biosynthesis and the control of cholesterol levels, HMG-CoA reductase inhibitors also exhibit anti-inflammatory activity. The methods of the invention are advantageous in that aerosol administration of a HMG-CoA reductase inhibitor allows localized administration of the inhibitor at the site of inflammation in the lung, thereby providing a higher effective dose at the site of inflammation. Localized administration in the lung is advantageous over systemic administration of a HMG-CoA reductase inhibitor because localized administration avoids exposure of high concentrations of a HMG-CoA reductase inhibitor to other tissues, which can lead to toxic side effects. Aerosol administration can also be advantageous in that a lower dose can be used because aerosol administration avoids the inefficient targeting of the HMG-CoA reductase inhibitor associated with systemic administration due to clearance in the liver or due to incomplete absorption in the intestine from oral administration. The compositions and methods are also advantageous in that they provide non-steroidal agents that are effective at decreasing inflammation in an inflammatory lung disease, which can thereby alleviate a sign or symptom of the disease.

As used herein, the term “3-hydroxy-3-methyl-glutaryl coenzyme A reductase” or “HMG-CoA reductase” is intended to refer to the enzyme that converts 3-hydroxy-3-methyl-glutaryl coenzyme A to mevalonate. Mevalonate is a precursor in the synthesis of cholesterol and isoprenoids. HMG-CoA reductase is found in eukaryotes and prokaryotes (Baltscheffsky et al., Biochem. Biophys. Acta 1337:113-122 (1997)). The HMG-CoA reductase to which inhibitors of the invention are directed is generally the enzyme present in eukaryotes, for example, the HMG-CoA reductase present in mammals, and in particular the HMG-CoA reductase present in primates, including human.

As used herein, the term “HMG-CoA reductase inhibitor” is intended to refer to a molecule that inhibits or reduces the activity of a HMG-CoA reductase. The HMG-CoA reductase inhibitors of the invention are active at inhibiting or reducing the activity of a HMG-CoA reductase in a mammal and, in particular, in a human. A HMG-CoA reductase inhibitor can be effective at inhibiting HMG-CoA reductase activity in different species, although a HMG-CoA reductase inhibitor can have differing inhibitory activities in different species. For example, the effective concentration of a HMG-CoA reductase inhibitor for mediating inhibitory activity can be lower in a human than in a mouse.

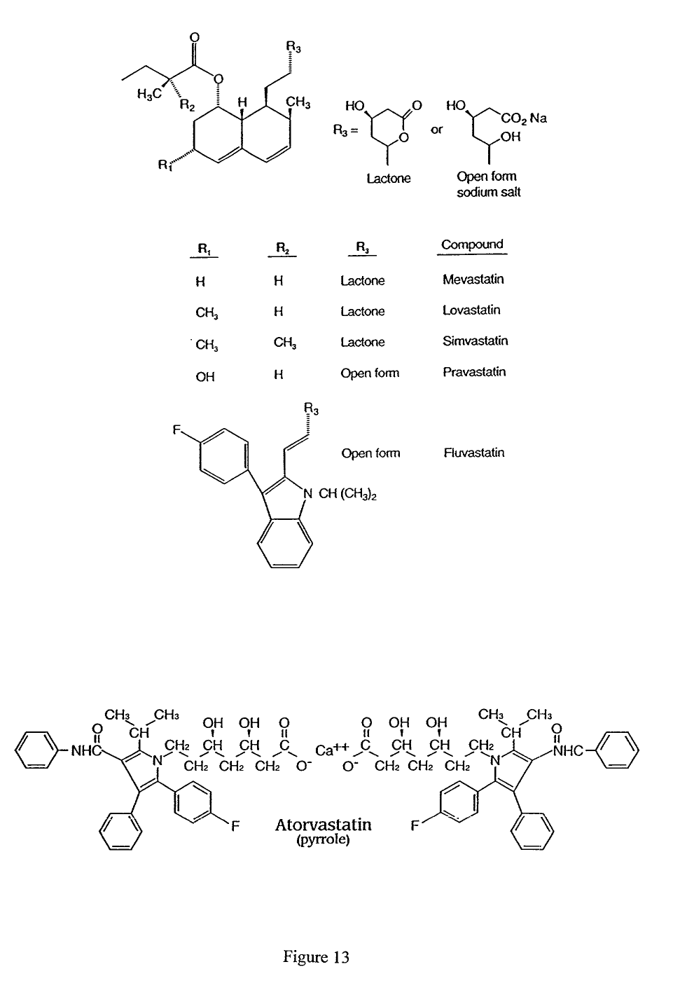

A HMG-CoA reductase inhibitor can be, for example, a competitive inhibitor of the enzyme such as a competitive inhibitor of the substrate HMG-CoA. An example of such an inhibitor is a statin, which is a class of compounds that are HMG-CoA reductase inhibitors. Statins include, for example, lovastatin, pravastatin, simvastatin, cerivastatin, fluvastatin, atorvastatin and mevastatin. The chemical structures of representative statins are shown in FIG. 13. Exemplary HMG-CoA reductase inhibitors are described, for example, in Desager and Horsmans (Clin. Pharmacokinet. 31:348-371 (1996)) and Hamelin and Turgeon (Trends Pharmacol. Sci. 19:26-37 (1998)).

As used herein, the term “inflammatory lung disease” is intended to refer to a disease associated with an inflammatory or immune response in the lung. Inflammatory lung diseases include, for example, acute lung injury, adult respiratory distress syndrome, asthma, emphysema, chronic bronchitis, cystic fibrosis, and interstitial lung disease such as interstitial pneumonitis, idiopathic fibrosis and interstitial fibrosis.

As used herein, the term “treating an inflammatory lung disease” is intended to refer to the alleviation of a sign or symptom of the inflammatory lung disease. Treating an inflammatory lung disease is intended to encompass a reduction in the onset or magnitude of a sign or symptom of an inflammatory lung disease.

As used herein, the term “aerosol formulation” is intended to refer to a pharmaceutical composition suitable for administration through the respiratory system or nasal passages. Examples of aerosol formulations are described below. Similarly, the term “aerosol administration” is intended to refer to a mode of administering an aerosol formulation to the respiratory system or nasal passages.

As used herein, the term “systemic formulation” is intended to refer to a pharmaceutical composition suitable for administration such that a drug or active agent is administered systemically throughout the body of an organism. Examples of systemic administration include oral administration or administration by injection.

The invention provides a composition of matter comprising an aerosol formulation of a HMG-CoA reductase inhibitor. In particular, the invention provides a composition of matter comprising a HMG-CoA reductase inhibitor that is a statin. Statins of the invention include, for example, lovastatin, pravastatin, simvastatin, cerivastatin, fluvastatin, atorvastatin and mevastatin.

The invention also provides a composition of matter comprising an aerosol formulation of a HMG-CoA reductase inhibitor, where the HMG-CoA reductase inhibitor is present at a concentration of less than 0.1 mg.

The invention also provides methods of treating a disease by administering a composition comprising a HMG-CoA reductase inhibitor. The methods of the invention are useful in that they provide an anti-inflammatory agent that is effective in alleviating a sign or symptom of the disease.

As disclosed herein, HMG-CoA reductase inhibitors are effective at reducing a sign or symptom of inflammation. The compositions of the invention are effective at inhibiting the adherence of leukocytes and the infiltration of inflammatory cells such as neutrophils into the lung in a mouse model of inflammatory lung disease. The compositions are also effective at inhibiting the production of cytokines during an inflammatory response, where the inhibition of cytokine production can result in a decrease in magnitude of the inflammatory response and alleviate a sign or symptom of a disease mediated by an inflammatory response. Therefore, the compositions are useful in methods of treating inflammatory lung disease.

As disclosed herein, HMG-CoA reductase inhibitors inhibit inflammation in the lung. For example, a HMG-CoA reductase inhibitor, lovastatin, was found to inhibit leukocyte cell adhesion stimulated by a phorbol ester or an integrin β₁-activating antibody (described further in Example I). Lovastatin was found to inhibit isoprenylation of rhoA, although MAP kinase signaling was not inhibited by lovastatin (see Examples II and III). The HMG-CoA reductase inhibitor lovastatin was also found to inhibit infiltration of lung by neutrophils in a mouse model of lung inflammation and asthma, which indicates that HMG-CoA reductase inhibitors are useful for decreasing an inflammatory response in lung (see Examples IV and VI). Lovastatin was also found to inhibit cytokine production in a mouse model of a pulmonary inflammatory response, which further indicates that HMG-CoA reductase inhibitors are useful for decreasing an inflammatory response in lung (described further in Example V).

The invention also provides a method of treating inflammatory lung disease by administering a pharmaceutical composition comprising an aerosol formulation of a HMG-CoA reductase inhibitor. The pharmaceutical composition can comprise, for example, a HMG-CoA reductase inhibitor that is a statin. The methods of the invention can employ, for example, a statin selected from the group consisting of lovastatin, pravastatin, simvastatin, cerivastatin, fluvastatin, atorvastatin and mevastatin.

The methods of the invention are particularly useful for treating inflammatory lung disease, including, for example, acute lung injury, adult respiratory distress syndrome, asthma, interstitial lung disease, emphysema, chronic bronchitis and cystic fibrosis.

Acute lung injury occurs when an insult to the lung causes an acute inflammatory reaction, which results in respiratory distress, hyoxemia and diffuse alveolar infiltrates and can lead to respiratory failure. Acute lung injury can occur with a variety of pulmonary insults, including, for example, sepsis and trauma. The extent of acute lung injury depends, for example, on the magnitude of initial damage, repeated insults such as persistent septicemia or retained necrotic and inflamed tissue, and added insults from treatment including barotrauma, hyperoxia and nosocomial infection.

Adult respiratory distress syndrome (ARDS) is a form of acute lung injury often seen in previously healthy patients. ARDS is characterized by rapid respiratory rates, a sensation of profound shortness of breath, sever hypoxemia not responsive to supplemental oxygen, and widespread pulmonary infiltrates riot explained by cardiovascular disease or volume overload. ARDS tends to follow a diverse array of systemic and pulmonary insults, although the majority of ARDS is associated with systemic or pulmonary infection, severe trauma, or aspirating gastric contents. The crucial stimulus to the development of ARDS is an inflammatory response to distant or local tissue injury. Disorders associated with ARDS include aspiration of gastric contents, fresh and salt water and hydrocarbons; central nervous system trauma, anoxia, seizures or increased intracranial pressure; drug overdose or reactions; hematologic alterations; infection including sepsis, pneumonia and tuberculosis; inhalation of toxins such as oxygen, smoke or corrosive chemicals; metabolic disorders such as pancreatitis; shock; and trauma such as fat emboli, lung contusion, sever nonthoracic trauma and cardiopulmonary bypass

Asthma is a disease of localized anaphylaxis, or atopy, and is characterized as an inflammatory disease. In some cases, asthma is triggered by exposure to allergens (allergic asthma), while in other cases, asthma is triggered independent of allergen stimulation (intrinsic asthma). Upon inhalation of an allergen by an asthmatic individual, an immune response is initiated, resulting in the release of mediators of hypersensitivity including histamine, bradykinin, leukotrienes, prostaglandins, thromboxane A₂ and platelet activating factor. The initial phase of the asthmatic response also results in the release of chemotactic factors that recruit inflammatory cells such as eosinophils and neutrophils. Clinical manifestations of these events include occlusion of the bronchial lumen with mucus, proteins and cellular debris; sloughing of the epithelium; thickening of the basement membrane; fluid buildup (edema); and hypertrophy of the bronchial smooth muscles.

Interstitial lung disease includes, for example, idiopathic fibrosis, interstitial fibrosis and interstitial pneumonitis. Interstitial pneumonitis, also known as hypersensitivity pneumonitis, results from inhaling diverse environmental antigens and chemicals. Symptoms of the disease include wheezing and dyspnea, and the disease is associated with infiltration of alveolar walls with lymphocytes, plasma cells, and other inflammatory cells. The disease can be an acute illness or can be present in a chronic form with pulmonary fibrosis upon progression to interstitial fibrotic disease with restrictive pattern on pulmonary function.

Chronic bronchitis is an inflammation of the bronchial tubes and can generally be manifested in two forms. “Simple chronic bronchitis” is correlated to exposure to environmental irritants, including occupational exposure to dust, grains and mining as well as cigarette smoking. Exposure to such environmental irritants is associated with inflammatory changes in the airways.

Another form of chronic bronchitis is “chronic obstructive bronchitis,” which is also strongly correlated with cigarette smoking. Patients exhibiting chronic obstructive bronchitis often have emphysema, which is similarly associated with cigarette smoking. Emphysema is associated with the chronic, progressive destruction of the alveolar structure and enlarged air spaces. The destruction of the alveolar structure is associated with proteases released by neutrophils (polymorphonuclear leukocyte; PMN) recruited into the lung by pulmonary alveolar macrophages. Symptoms of emphysema include undue breathlessness upon exertion.

Cystic fibrosis is a lethal genetic disease characterized by abnormally viscous mucous secretions, which lead to chronic pulmonary disease. Defective chloride ion secretion occurs in cystic fibrosis due to mutations in an epithelial cell chloride ion channel, the cystic fibrosis transmembrane regulator (CFTR). Disease progression is often marked by gradual decline in pulmonary function. The major source of morbidity in cystic fibrosis patients is pulmonary disease associated with chronic and recurrent bacterial infections and the detrimental cumulative long-term effects of the resulting inflammatory response on the pulmonary tissue.

As effector cells of the immune system, leukocytes are capable of synthesizing and releasing a plethora of bioactive molecules, including oxidants, eicosanoids, proteases, and cytokines. Although critical for host defense and repair, under some circumstances, leukocytes and leukocyte-derived products may contribute to endothelial and tissue injury. In order for leukocytes to cause vascular or tissue damage, they must first adhere to the endothelium and then exit the blood stream to gain access to extravascular tissue. Leukocyte and endothelial adhesion molecules are necessary not only for adherence and emigration, but they participate in several other leukocyte functions, such as the transduction of signals regulating gene expression and cell survival. Blockade of leukocyte-endothelial adhesive interactions has helped to elucidate the role of leukocytes in the genesis of vascular and tissue injury in experimental models of inflammatory and immune disorders. Moreover, adhesion molecules have emerged as important targets of therapy in a broad spectrum of human diseases.

The most convincing evidence of leukocyte-mediated endothelial injury comes from studies of ischemia-reperfusion (I-R) injury. I-R injury is potentially involved in a wide spectrum of disease processes, including myocardial infarction, stroke, mesenteric and peripheral vascular ischemia, limb and digit re-implantation, compartment syndromes, thermal injury, and circulatory shock. A major component of I-R injury is believed to occur during the reperfusion phase when an inflammatory response triggers neutrophil adhesion to endothelium and transmigration to tissue and subsequent neutrophil-mediated endothelial and tissue injury. Blockade of leukocyte and endothelial adhesion molecules has been demonstrated to attenuate vascular injury, manifested by plasma leakage or thrombosis, and tissue damage in a wide variety of animal models of I-R injury. Clinical trials evaluating the efficacy of adhesion molecule blockade in reducing organ damage are underway in hemorrhagic shock, stroke, and myocardial infarction.

Blockade of leukocyte or endothelial adhesion molecules has also been demonstrated to be of benefit in models of chronic inflammatory-immune diseases, such as allograft rejection, multiple sclerosis, asthma, arthritis, and inflammatory bowel disease. Recently, a new perspective has emerged that views atherosclerosis as a chronic inflammatory-immune process. Like other chronic inflammatory reactions, experimental and human atherosclerotic lesions exhibit an accumulation of mononuclear phagocytes as well as lymphocytes. Mononuclear phagocytes produce multiple mediators, such as growth factors, proteases, and cytokines, that could contribute to the initiation and progression of the arterial lesions by inducing endothelial dysfunction with altered permeability or vasoreactivity; accumulating lipid and becoming foam cells; promoting smooth muscle cell migration and proliferation; and destabilizing the plaque. Consequently, there has been a great deal of interest in elucidating the mechanisms of mononuclear phagocyte recruitment into the arterial wall. Upregulated expression of VCAM-1, ICAM-1, E-selectin, and P-selectin has been demonstrated on arterial luminal endothelium in human and experimental lesions. Limited studies have tested anti-adhesion therapy in atherosclerosis-related models. Blockade of VLA-4 and CD18 or ICAM-1 and LFA-1 were reported to attenuate intimal smooth muscle cell hyperplasia in rat and rabbit models of arterial injury. More direct evidence for a role of endothelial cell adhesion molecules in atherogenesis comes from gene-targeted mice with deficiencies of adhesion molecules in which arterial lesion formation is reduced during hyperlipidemia-induced atherogenesis.

These in vivo studies in animal models confirm the importance of leukocyte and endothelial adhesion molecules in the recruitment of mononuclear phagocytes into the arterial wall where they develop into the foam cells that constitute the fatty streak. They also suggest a role for leukocytes in intimal smooth muscle cell accumulation.

HMG-CoA reductase inhibitors are also effective at inhibiting inflammation in sites other than lung. For example, systemic administration of a HMG-CoA reductase inhibitor can be used to inhibit inflammation, including sites other than the lung. In particular, systemic administration of a HMG-CoA reductase inhibitor can be advantageous when direct application of a HMG-CoA reductase inhibitor at the site of inflammation is not practical. For example, lovastatin was found to have anti-inflammatory activity during ischemia and reperfusion of mouse hind limb, indicating that HMG-CoA reductase inhibitors are effective at inhibiting inflammation (Example VII). Therefore, systemic administration of a HMG-CoA reductive inhibitor can be useful for treating inflammatory lung diseases as disclosed herein. In addition, systemic administration of a HMG-CoA reductase inhibitor can be useful for treating ischemia-reperfusion injury, including stroke, hemorrhagic shock, artherosclerosis, myocardial infarction, mesenteric and peripheral vascular ischemia, limb and digit re-implantation, compartment syndromes and thermal injury. Systemic administration of a HMG-CoA reductase inhibitor can also be useful in treating chronic inflammatory immune diseases such as, allograft rejection, multiple sclerosis, arthritis, inflammatory bowel disease and artherosclerosis.

The invention also provides compositions comprising two or more HMG-CoA reductase inhibitors. The invention additionally provides methods of using a composition comprising two or more HMG-CoA reductase inhibitors to treat inflammation. As described below, the dosage for a given HMG-CoA reductase inhibitor can be readily determined by one skilled in the art. Similarly, one skilled in the art can adjust the dosage of each HMG-CoA reductase inhibitor so that the combination provides an effective dose for treating inflammation.

Methods of the invention directed to treating an inflammatory lung disease, for example, acute lung injury, adult respiratory distress syndrome, asthma, emphysema, chronic bronchitis, cystic fibrosis, or interstitial lung disease such as interstitial pneumonitis, idiopathic fibrosis or interstitial fibrosis, involve the administration of a HMG-CoA reductase inhibitor in a pharmaceutical composition. A HMG-CoA reductase inhibitor is administered to an individual as a pharmaceutical composition comprising a HMG-CoA reductase inhibitor and a pharmaceutically acceptable carrier. Pharmaceutically acceptable carriers are well known in the art and include aqueous solutions such as physiologically buffered saline or other solvents or vehicles such as glycols, glycerol, oils such as olive oil or injectable organic esters.

A pharmaceutically acceptable carrier can contain physiologically acceptable compounds that act, for example, to stabilize the HMG-CoA reductase inhibitor or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the HMG-CoA reductase inhibitor and on the particular physico-chemical characteristics of the specific HMG-CoA reductase.

One skilled in the art would know that a pharmaceutical composition comprising a HMG-CoA reductase inhibitor can be administered to a subject by various routes including, for example, orally or parenterally, such as intravenously (i.v.), intramuscularly, subcutaneously, intraorbitally, intracapsularly, intraperitoneally (i.p.), intracisternally, intra-articularly or by passive or facilitated absorption through the skin using, for example, a skin patch or transdermal iontophoresis, respectively. Thus, a HMG-CoA reductase inhibitor can be administered systemically by injection, intubation, or orally, or can be administered locally by topical application, the latter of which can be passive, for example, by direct application of an ointment or powder, or active, for example, using a nasal spray or inhalant.

Administration of a HMG-CoA reductase inhibitor by inhalation is a particularly useful means of treating an individual having an inflammatory lung disease. One skilled in the art would recognize that a HMG-CoA reductase inhibitor can be suspended or dissolved in an appropriate pharmaceutically acceptable carrier and administered, for example, directly into the lungs using a nasal spray or inhalant.

A pharmaceutical composition comprising a HMG-CoA reductase inhibitor can be administered as an aerosol formulation. An aerosol formulation contains a HMG-CoA reductase inhibitor dissolved, suspended or emulsified in a propellant or a mixture of solvent and propellant and is intended for administration as a nasal spray or inhalant. The aerosol is administered through the respiratory system or nasal passages.

An aerosol formulation used for nasal administration is generally an aqueous solution designed to be administered to the nasal passages in drops or sprays. Nasal solutions are generally prepared to be similar to nasal secretions and are generally isotonic and slightly buffered to maintain a pH of about 5.5 to about 6.5, although pH values outside of this range can additionally be used. Antimicrobial agents or preservatives can also be included in the formulation.

An aerosol formulation used for inhalations and inhalants is designed so that the HMG-CoA reductase inhibitor is carried into the respiratory tree of the patient administered by the nasal or oral respiratory route. Inhalation solutions can be administered, for example, by a nebulizer. Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the HMG-CoA reductase inhibitor. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Reminaton's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Representative halocarbon propellants are shown, for example, in Table 1. In Table 1, “cy” denotes that the propellant is a cyclic compound in which the end terminal covalent bonds of the structures shown are the same so that the end terminal groups are covalently bonding together.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as the ethers shown in Table 1.

The HMG-CoA reductase inhibitor can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, supra, 1990; Purewal et al., U.S. Pat. No. 5,776,434). These aerosol components can serve to stabilize the formulation and lubricate valve components. Representative aerosol components, are shown, for example, in Table 2.

TABLE 1
Halocarbon Propellants

| trichlorofluoromethane | |
|---|---|
| dichlorodifluoromethane | |
| dichlorotetrafluoroethane | |
| difluoroethane | |
| monochlorodifluoromethane | |
| monochlorodifluroethane | |
| 1,1,1,2-tetrafluoroethane | |
| heptafluoropropane | |
| 2-bromo-2-chloro-1,1,1-trifluoroethane | |
| 2-chloro-1-(difluoromethoxy)-1,1,2-trifluoroethane | |
| 2-chloro-2-difluoromethoxy)-1,1,1-trifluoroethane | |
| CF₃—CHF—CF₂H | CF₂H—CF₂—CH₂Cl |
| CF₃—CH₂—CF₂H | CF₃—CF₂—CF₂—CH₃ |
| CF₃—CHF—CF₃ | CF₃—CF₂—CF₂—CF₂H |
| CF₃—CH₂—CF₃ | CF₃—CHF—CHF—CF₃ |
| CF₃—CHCl—CF₂Cl | CF₃—O—CF₃ |
| CF₃—CHCl—CF₃ | CF₃—O—CF₂H |
| cy-C(CF₂)—CHCl | CF₂H—O—CF₂H |
| CF₃—CHCl—CH₂Cl | CF₂H—O—CFH₂ |
| CF₃—CHF—CF₂Cl | CF₃—O—CH₃ |
| CF₃—CHCl—CFHCl | CF₃—O—CF₂—CF₂H |
| CF₃—CFCl—CFHCl | CF₃—O—CF₂—O—CF₂—CF₃ |
| CF₃—CF₂—CF₂H | cy-CF₂—CF₂—O—CF₂— |
| CF₃—CF₂—CH₃ | cy-CHF—CF₂—O—CF₂— |
| CF₂H—CF₂—CFH₂ | cy-CH₂—CF₂—O—CF₂— |
| CF₃—CF₂—CFH₂ | cy-CF₂—O—CF₂—O—CF₂— |
| CF₃—CF₂—CH₂Cl | CF₃—O—CF₂—Br |
| CF₂H—CF₂—CH₃ | CF₂H—O—CF₂—Br |

The aerosol formulation can be packaged under pressure and can be formulated as an aerosol using solutions, suspensions, emulsions, powders and semisolid preparations. A solution aerosol consists of a solution of an active ingredient such as a HMG-CoA reductase inhibitor in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient HMG-CoA reductase inhibitor and a propellant and can include any combination of solvents and preservatives or antioxidants.

An aerosol formulation can also be a dispersion or suspension. A suspension aerosol formulation will generally contain a suspension of a HMG-CoA reductase inhibitor and a dispersing agent. Dispersing agents useful in the invention include, for example, sorbitan trioleate, oleyl alcohol, oleic acid, lecithin and corn oil. A suspension aerosol formulation can also include lubricants and other aerosol components such as those shown in Table 2.

TABLE 2
Aerosol Components

mineral oil
corn oil
olive oil
cotton seed oil
sunflower oil
sorbitan trioleate (SPAN 85)
sorbitan mono-oleate (SPAN 80)
sorbitan monolaurate (SPAN 20)
polyoxyethylene (20) sorbitan monolaurate (TWEEN 20)
polyoxyethylene (20) sorbitan mono-oleate (TWEEN 80)
lecithin (EPIKURON, EPIKURON 200)
oleyl polyoxyethylene (2) ether (BRIJ 92)
stearyl polyoxyethylene (2) ether (BRIJ 72)
lauryl polyoxyethylene (4) ether (BRIJ 30)
oleyl polyoxyethylene (2) ether (GENAPOL 0-020)
oxyethylene and oxypropylene copolymers (SYNPERONIC)
oleic acid
synthetic lecithin
diethylene glycol dioleate
tetrahydrofurfuryl oleate
ethyl oleate
isopropyl myristate
glyceryl trioleate
glyceryl monolaurate
glyceryl mono-oleate
glyceryl monostearate
glyceryl monoricinoleate
cetyl alcohol
stearyl alcohol
polyethylene glycol 400
cetyl pyridinium chloride

An aerosol formulation can similarly be formulated as an emulsion. An emulsion can include, for example, an alcohol such as ethanol, a surfactant, water and propellant, as well as the active ingredient HMG-CoA reductase inhibitor. The surfactant can be nonionic, anionic or cationic. One example of an emulsion can include, for example, ethanol, surfactant, water and propellant. Another example of an emulsion can include, for example, vegetable oil, glyceryl monostearate and propane.

An aerosol formulation containing a HMG-CoA reductase inhibitor will generally have a minimum of 90% of the particles in inhalation products between about 0.5 and about 10 μm to maximize delivery and deposition of the HMG-CoA reductase inhibitor to respiratory fluids. In particular, the particle size can be from about 3 to about 6 μm.

A pharmaceutical composition comprising a HMG-CoA reductase inhibitor also can be incorporated, if desired, into liposomes, microspheres or other polymer matrices (Gregoriadis, Liposome Technology, Vols. I to III, 2nd ed., CRC Press, Boca Raton Fla. (1993)). Liposomes, for example, which consist of phospholipids or other lipids, are nontoxic, physiologically acceptable and metabolizable carriers that are relatively simple to make and administer.

In order to treat an individual having an inflammatory lung disease to alleviate a sign or symptom of the disease, a HMG-CoA reductase inhibitor should be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of a HMG-CoA reductase inhibitor required to obtain an effective dose in a subject depends on many factors, including the particular inflammatory lung disease being treated, the age, weight and general health of the subject and the particular HMG-CoA reductase inhibitor being administered as well as the route of administration and the number of treatments to be administered. In view of these factors, the skilled artisan would adjust the particular dose so as to obtain an effective dose for treating an individual having an inflammatory lung disease.

The effective dose of a HMG-CoA reductase inhibitor will depend on the particular HMG-CoA reductase used and the mode of administration, as well as the weight of the individual being treated. The dosages described herein are generally those for an average adult. The dose will generally range from about 0.001 mg to about 3500 mg. Unlike the use of HMG-CoA reductase inhibitors for the treatment of high cholesterol, the present invention is directed to methods of treating inflammation, which can be treated as an acute response. Therefore, the HMG-CoA reductase inhibitors of the invention can be administered at high doses relative to those given for lowering cholesterol levels. The dose will generally be at least about 10 mg per day, at least about 100 mg per day, at least about 200 mg per day, at least about 250 mg per day, at least about 300 mg per day, at least about 400 mg per day, or at least about 500 mg per day, and can be at least about 1000 mg per day. The use of relatively high doses is particularly useful for systemic administration of a HMG-CoA reductase inhibitor.

When administering high doses of a HMG-CoA reductase inhibitor, one skilled in the art can monitor for any possible adverse side effects. Methods of monitoring adverse side effects of a HMG-CoA reductase inhibitor, including myopathy, are described, for example, in Thibault et al., Clinical Cancer Res. 2:483-491 (1996). One skilled in the art can monitor for any adverse side effects and, if necessary, adjust the dosage to minimize adverse side effects while optimizing the effectiveness of treating an inflammatory lung disease.

For administration in an aerosol formulation, the dose of HMG-CoA reductase inhibitor can generally be lower than the dose used for systemic administration. For example, a HMG-CoA reductase inhibitor can be administered at a dose lower than about 10 mg per day, generally lower than about 1 mg per day, and in particular lower than about 0.1 mg day. The HMG-CoA reductase inhibitor can be administered at a dose of less than 0.1 mg per day, for example, about 0.09 mg per day or less, about 0.08 mg per day or less, about 0.07 mg per day or less, about 0.06 mg per day or less, about 0.05 mg per day or less, about 0.04 mg per day or less, about 0.03 mg per day or less, about 0.02 mg per day or less, or about 0.01 mg per day or less.

The concentration of a HMG-CoA reductase inhibitor in a particular formulation will depend on the mode and frequency of administration. A given daily dosage can be administered in a single dose or in multiple doses so long as the HMG-CoA reductase inhibitor concentration in the formulation results in the desired daily dosage. For example, a given formulation can contain a HMG-CoA reductase inhibitor at a concentration of about 0.09 mg, about 0.08 mg, about 0.07 mg, about 0.06 mg, about 0.05 mg, about 0.04 mg, about 0.03 mg, about 0.02 mg or about 0.01 mg. A given formulation can also contain a HMG-CoA reductase inhibitor at a concentration of about 0.005 mg, about 0.002 mg or about 0.001 mg. One skilled in the art can adjust the amount of HMG-CoA reductase inhibitor in the formulation to allow administration of a single dose or in multiple doses that provide the desired concentration of HMG-CoA reductase inhibitor over a given period of time. For example, the formulation can be adjusted to allow administration of a single dose or multiple doses that provides less than 0.1 mg per day of a HMG-CoA, reductase inhibitor.

The invention also provides a method of treating an inflammatory lung disease, comprising administering a pharmaceutical composition comprising an aerosol formulation of a HMG-CoA reductase inhibitor, wherein the HMG-CoA reductase inhibitor is administered in an aerosol formulation at an amount or total dosage lower than in a systemic formulation. For example, the amount or total dosage administered as an aerosol formulation can be administered at a dosage lower than the amount or total dosage that would be administered by other modes of administration such as systemic, intraperitoneal or oral administration.

The amount or total dosages for systemic administration of a HMG-CoA reductase inhibitor are well known in the art as described, for example, in Physician's Desk Reference 52nd ed., Medical Economics Company (1998). It is known that the dosage of a given HMG-CoA reductase varies with different inhibitors. Therefore, the dosage for a particular HMG-CoA. reductase in an aerosol formulation is adjusted so that it is less than the dosage of the same inhibitor administered systemically.

The invention also provides a method of treating an inflammatory lung disease, comprising administering a pharmaceutical composition comprising an aerosol formulation of a HMG-CoA reductase inhibitor, wherein the HMG-CoA reductase inhibitor is administered at an amount or total dosage lower than the amount or total dosage of the HMG-CoA reductase inhibitor administered in a systemic formulation.

In an individual suffering from an inflammatory lung disease, in particular a more severe form of the disease, administration of a HMG-CoA reductase inhibitor can be particularly useful when administered in combination, for example, with a conventional agent for treating such a disease. The skilled artisan would administer a HMG-CoA reductase inhibitor, alone or in combination with a second agent, based on the clinical signs and symptoms exhibited by the individual and would monitor the effectiveness of such treatment using routine methods such as pulmonary function determination, radiologic, immunologic or, where indicated, histopathologic methods.

A HMG-CoA reductase inhibitor can be administered in combination with one or more other compounds that are effective at treating inflammation such as other anti-inflammatory agents. A HMG-CoA reductase inhibitor can be administered in combination with steroidal anti-inflammatory agents including corticosteroids, for example, dexamethasone, beclomethasone, fluticasone, triamcinolone and budesonide. A HMG-CoA reductase inhibitor can also be administered in combination with non-steroidal anti-inflammatory agents such as aspirin (acetylsalicylic acid), indomethacin, ibuprofen, naproxen, diclofenac, sulindac, oxaprozin, diflunisal, bromfenac, piroxicam, etodolac and fenoprofen. A HMG-CoA reductase inhibitor can additionally be used in combination with a leukotriene synthesis inhibitor or antagonist, including leukotriene receptor antagonists such as montelukast, pranlukast, zafirlukast and tomelukast, or with 5-lipoxygenase inhibitors such as zileuton (Drazen et al., N. Engl. J. Med. 340:197-206 (1999)). When a HMG-CoA reductase inhibitor is used with another anti-inflammatory agent, the HMG-CoA reductase inhibitor can generally be administered at a lower dosage. For example, a HMG-CoA reductase inhibitor can be administered at a dose of less than 0.1 mg per day in combination with another anti-inflammatory agent.

When a HMG-CoA reductase inhibitor is administered in combination with one or more other anti-inflammatory agent, the HMG-CoA reductase inhibitor and other anti-inflammatory agent can be co-administered in the same formulation. Alternatively, the HMG-CoA reductase inhibitor and other anti-inflammatory agent can be administered simultaneously in separate formulations. In addition, the HMG-CoA reductase inhibitor can be administered in separate formulations, where the separate formulations are not administered simultaneously but are administered during the same period of treatment, for example, during a daily or weekly period of treatment.

It is understood that modifications which do not substantially affect the activity of the various embodiments of this invention are also provided within the definition of the invention provided herein. Accordingly, the following examples are intended to illustrate but not limit the present invention.

### EXAMPLE I

### Inhibition of Cell Adhesion by HMG-CoA Reductase Inhibitors

This example demonstrates that a HMG-CoA reductase inhibitor, lovastatin, blocks cell adhesion in vitro.

The effect of lovastatin on cell adherence was determined. Briefly, Jurkat and U937 cells (American Type Culture Collection; Manassas Va.) were maintained in Earles Modified Eagles Medium (EMEM; M. A. Bioproducts, Walkersville, Md.) supplemented with 10% fetal bovine serum (FBS; Hyclone Laboratories Inc.; Logan Utah), 5 mM glutamine (Life Technologies Inc.; Gaithersburg Md.), 5 mM sodium pyruvate (Life Technologies), and 5 mM non-essential amino acids (Life Technologies). Lovastatin (Calbiochem; San Diego Calif.) was converted to open acid form before use. Briefly, 25 mg lovastatin was suspended in 0.5-1 ml ethanol, and 3 ml of 0.1 M NaOH was added. The solution was heated to 50° C. for two hours. To this heated solution, 4.5 ml of an aqueous solution containing 81 mM NaH₂PO₄ and 15 mM NaH₂PO₄ was added. The solution was heated to 40° C. for 30 min and the pH was adjusted to 7.3 with concentrated HCl. The final concentration was determined by UV spectroscopy (ε₂₃₁ =21,490 M⁻¹cm⁻¹). The stock solution was frozen in small aliquots.

Cell adhesion of Jurkat cells and U937 cells to fibronectin (FN) or laminin (LN), respectively, was measured. Human FN (1.25 μg/ml)(Collaborative Research Inc.; Bedford Mass.) or human LN (100 μg/ml)(Life Technologies) was coated onto 96-well PRO-BIND assay plate (Falcon; Becton Dickinson; Lincoln Park N.J.) by incubating overnight at 4° C. The plate was then blocked with 3% bovine serum albumin (BSA) in phosphate buffered saline (PBS) at room temperature for one hour. Immediately before use, plates were washed three times with PBS.

Jurkat cells and U937 cells were pretreated with or without lovastatin for 2.5 days in EMEM. After centrifugation for seven min at 300 xg, cells were resuspended in 1 ml phenol red-free medium and then labeled by incubation with 5 μl of the fluorescent dye calcein-AM (1 mg/ml in DMSO; Molecular Probes; Eugene Oreg.) for 30 min at room temperature in the dark. The cells were then washed twice with phenol red-free medium. After incubation with phorbol 12-myristate 13--acetate (PMA) (100 ng/ml) (Sigma; St. Louis Mo.), which is an activator of protein kinase C (PKC), or MAb 8A2 (2 μg/ml) for 30 min in control medium at room temperature, cells (˜1×10⁵/well) were added to triplicate wells. After incubation for 30 min at 37° C., the total cells in the well were analyzed using a fluorescence plate reader (PerSeptive Biosystem; Framingham Mass.). Unbound cells were removed by washing the plate three times with phenol red-free medium, and the plate was then reanalyzed to determine fluorescence of bound cells. After subtraction of background, the percent adherence was calculated as the emission at 530 nm of bound cells divided by the absorption of total cells.

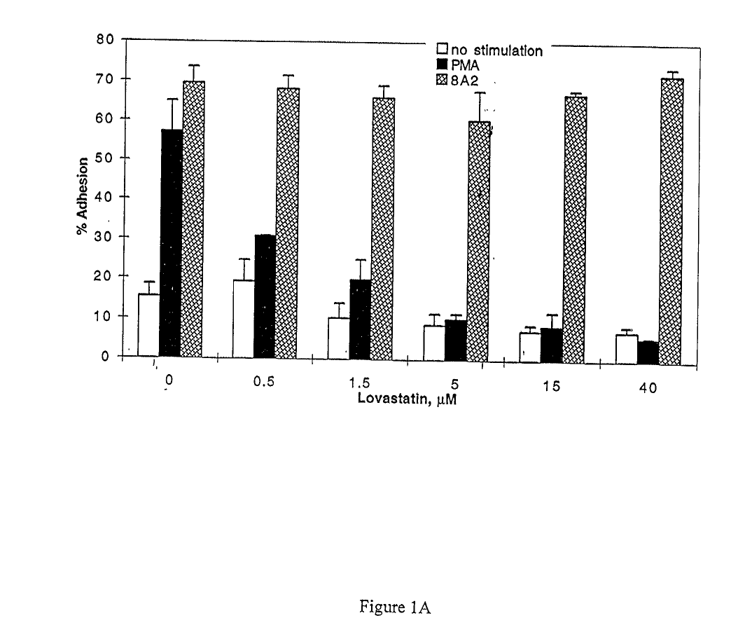

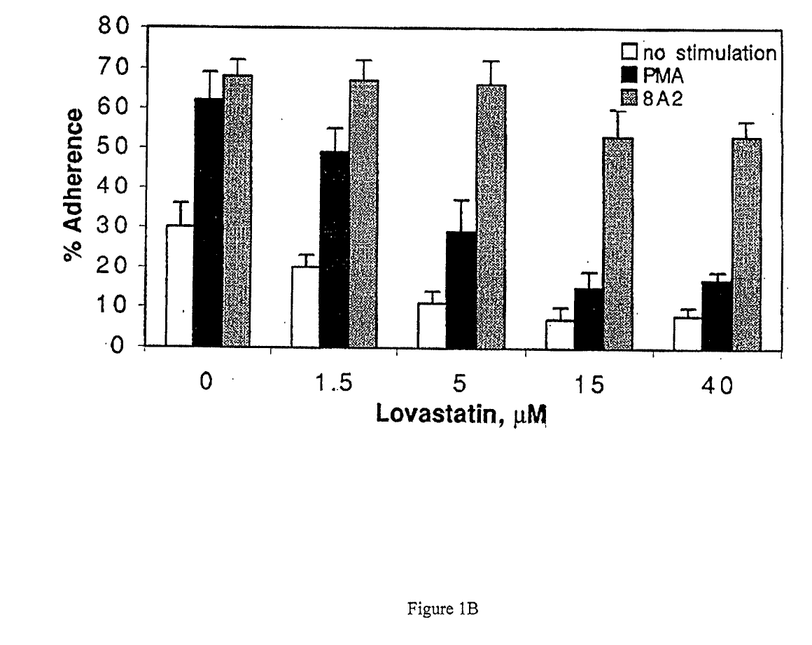

Both PMA and the β₄-activating mAb 8A2 markedly stimulated Jurkat cell adherence to FN (FIG. 1A), and this stimulated adhesion was blocked by MAb to the α₄ subunit (antibody HP1/2, provided by Dr. Roy Lobb; Biogen; Cambridge Mass.; Escudero et al., J. Am. Soc. Nephrol. 9:1881-1891 (1998)) or β₁ subunit (antibody P4C10; Life Technologies) (antibody 5D1; provided by B. Schwartz; University of Washington, Seattle Wash.) but not the α₅ subunit (antibody P1D6; Life Technologies). To determine the effect of lovastatin on cell adhesion, Jurkat cells were exposed to varying concentrations of lovastatin for 2.5 days prior to the adherence assay. As shown in FIG. 1A lovastatin at a concentration greater than 5 μM nearly completely inhibited PMA-stimulated Jurkat cell adherence to FN with an IC₅₀ of 1-2 μM. Interestingly, lovastatin had no significant effect on mAb 8A2-stimulated Jurkat cell adherence to FN. The insert in FIG. 1A shows the curve for lovastatin inhibition of PMA-stimulated Jurkat cell adherence to FN with a calculated IC₅₀ of 1 μM. Values represent the mean ±SE of triplicate wells. Similar results were found in two other experiments.

PMA and 8A2 also stimulated U937 cell adherence to LN (FIG. 1B), and this stimulated adherence was blocked by mAB to the α₆ subunit (GoH3; Kamiya Biomedical Co.; Seattle Wash.). Lovastatin also inhibited PMA-stimulated adherence but not mAb 8A2-stimulated adherence of U937 cells to LN (FIG. 1B). Addition of lovastatin at the time of the adhesion assay, instead of pretreating with lovastatin, was without effect.

These results show that lovastatin inhibits cell adhesion.

### EXAMPLE II

### Effect of Lovastatin on the MAP Kinase Signaling Pathway

This example demonstrates the role of lovastatin in signaling pathways.

Since lovastatin inhibited cell adhesion in response to the PKC inhibitor PMA, the role of lovastatin in signaling pathways, in particular pathways involving PKC mediated signaling, was investigated. The effect of PMA-induced MAP kinase kinase (MAPKK) activation was analyzed to determine whether the effect of lovastatin on PMA-induced adherence was due to blockade of all PKC mediated signaling. The predominant MAP kinases in T cells are ERK1 and ERK2 (Whitehurst et al., J. Immunol. 148:3230-3237 (1992)). PMA activates PKC and ultimately results in threonine and tyrosine phosphorylation of ERK1 and ERK2.

Phosphorylation of ERK1 in control and lovastatin-treated cells was monitored by immunoblot analysis with anti-phosphorylated ERK1, an antibody that recognizes only the phosphorylated form of ERK1. Briefly, whole cell lysate was prepared by rapidly pelleting 1.0×10⁶ cells and lysing in 1× sodium dodecyl sulfate (SDS) sample buffer. Proteins from whole cell lysate were subjected to SDS-polyacrylamide gel electrophoresis (SDS-PAGE) and transferred electrophoretically to nitrocellulose paper (Intermountain Scientific Corporation; Kayville Utah). After the transfer, nitrocellulose membranes were blocked overnight at 4° C. in 5% nonfat milk in a Tris-buffered saline of 0.05% TWEEN 20 (TBST) and then immunoblotted with the indicated antibodies. ERK1 immunoblots were prepared by sequential incubation with anti-phosphorylated ERK1 (New England Biolabs Inc.; Beverly Mass.) at room temperature for three hours and with HRP-conjugated goat anti-rabbit antibody for one hour. After five washings with TBST, reactive proteins were visualized by chemiluminescence using the luminol reagent (Santa Cruz Biotechnology, Inc.; Santa Cruz Calif.)

Jurkat cells were pretreated with lovastatin for 2.5 days in EMEM and then treated with or without PMA (100 ng/ml) for 30 min. Cells were lysed and the lysates were subjected to immunoblot analysis for phosphorylated ERK1. The immunoblot analysis shows that PMA still activated this MAP kinase (MAPK) cascade even at lovastatin concentrations up to 40 μM. Thus, lovastatin does not inhibit PMA-stimulated MAPK phosphorylation. This result indicates that treatment with lovastatin does not disable all components of PMA-stimulated signaling but specifically blocks one or more elements in the pathway leading to increased adhesiveness. These results also indicate that the MAPK cascade does not play a role in adhesion induced by post-receptor events.

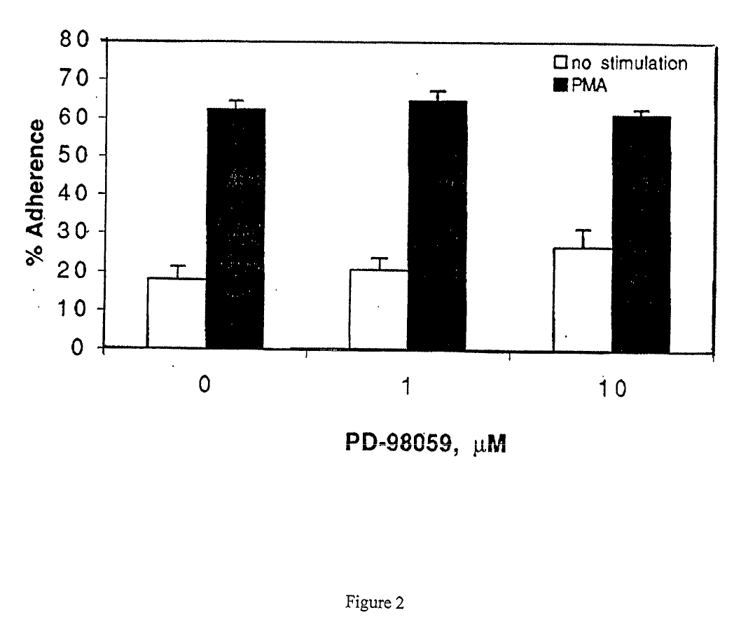

To further confirm that the MAPK cascade does not play a role in PMA-stimulated cell adherence, cells were pretreated with a specific inhibitor of MAPKK (MEK1) prior to the adhesion assay. For the inhibition experiments, Jurkat cells were pre-incubated at room temperature with the MEK-1 inhibitor PD-98059 (0-10 μM) (Calbiochem) for 30 min in medium prior to stimulation with PMA (Dudley et al. Proc. Natl. Acad. Sci. USA 92:7686-7689 (1995)). The adherence assay was performed as described in Example I. Although immunoblot analysis demonstrated that ERK1 phosphorylation was completely blocked by the MEK-1 inhibitor, treatment with PD-98059 (10 μM) did not inhibit PMA-stimulated Jurkat cell adherence (FIG. 2).

These results show that the MAPK cascade is not involved in PMA-stimulated cell adherence to fibronectin and that lovastatin does not affect MAPK signaling.

### EXAMPLE III

### Lovastatin Inhibits Isoprenylation of RhoA

This example demonstrates that geranylgeranylated proteins regulate PMA-stimulated cell adhesion and that lovastatin inhibits isoprenylation of RhoA.

Since mevalonic acid (MVA) can be converted to two kinds of isoprenyl groups, geranylgeranyl and farnesyl, the effect of lovastatin on stimulated leukocyte adherence could be explained by lowering of the cellular pool of either geranylgeranyl pyrophosphate (GGPP) or farnesyl pyrophosphate (FPP) or both. In order to determine which isoprenyl group was involved, the internal supply of both lipids was depleted by lovastatin treatment, and the effect of GGPP or FPP on cell adherence was determined. Since GGPP can be converted from all trans-geranylgeraniol (GGOH) in cells and FPP can be converted from trans, trans-farnesol (FOH), the GGOH and FOH forms, which are more cell-permeable, were used. The all-trans GGOH and all-trans FOH forms were used because the isoprenyl groups that modify small G-proteins are all trans isomer.

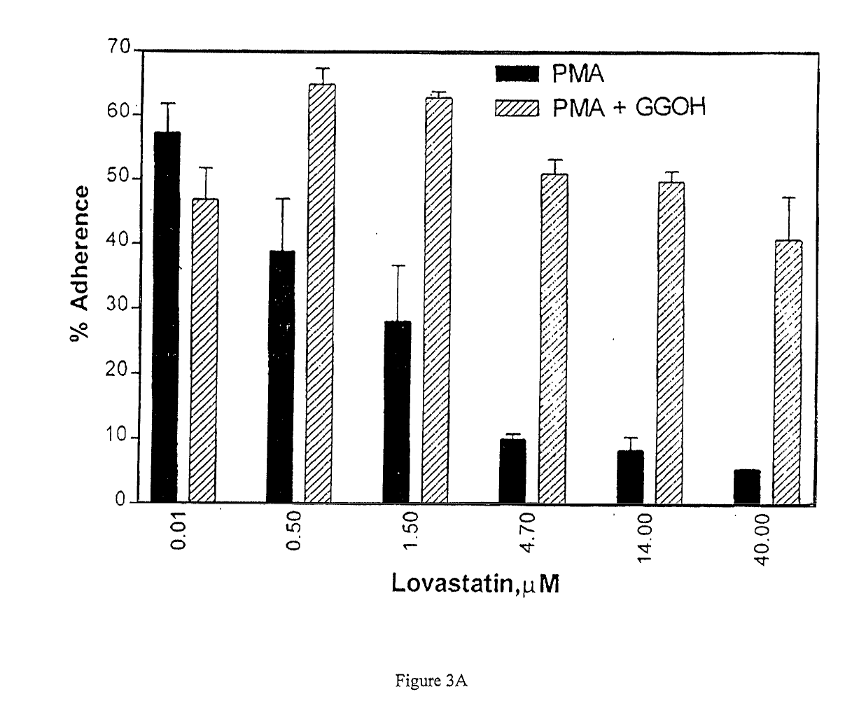

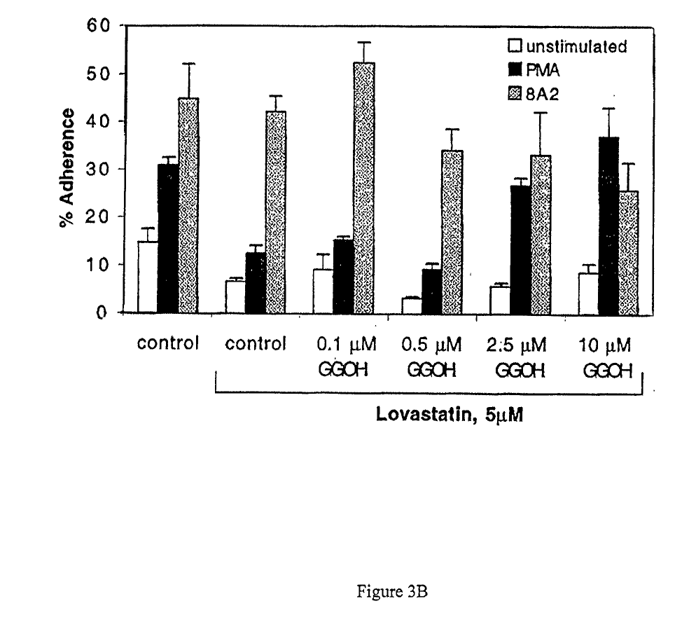

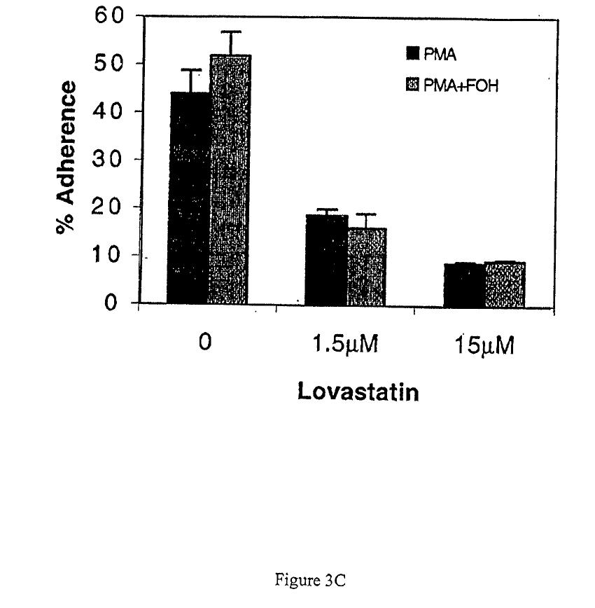

Jurkat cells were co-incubated with all trans-geranylgeraniol (GGOH) (American Radiolabeled Chemicals; St. Louis Mo.) or trans, trans-farnesol (FOH) (Sigma) together with lovastatin for 2.5 days prior to assay. When cells were treated with lovastatin and GGOH or FOH, GGOH (FIGS. 3A and 3B), but not FOH (FIG. 3C), reversed the inhibitory effect of lovastatin. It required 2.5 μM of GGOH to rescue Jurkat cell from the inhibitory effect of 10 μM lovastatin (FIG. 3B). This result indicates that PMA-stimulated cell adhesion involves at least one isoprenylated protein and that this protein is geranylgeranylated but not farnesylated.

Ras family members, H-Ras, R-Ras and RhoA have been implicated in cell adhesion (Zhang et al., Cell 85:61-69 (1996); Hughes et al., Cell 88:521-530 (1997); Ridley et al., Cell 70:389-399 (1992); Narumiya et al. FEBS Lett. 410:68-72 (1997)). RhoA is a geranylgeranylated protein (Hori et al., Oncogene 6:515-522 (1991)). The effect of lovastatin on RhoA isoprenylation and the correlation between RhoA isoprenylation and cell adhesion was examined.

Jurkat cells were pretreated with lovastatin for 2.5 days, then treated with or without PMA (100 ng/ml) for 30 min. Cell lysates were subjected to immunoblot analysis to assess RhoA electrophcoretic mobility as a measurement of isoprenylation. For RhoA blots, membranes were sequentially incubated with rabbit anti-RhoA antibody (Santa Cruz Biotechnology, Inc.) in TBST at room temperature for three hours, washed with TBST five times, incubated with goat anti-rabbit IgG (H+L) conjugated with horse radish peroxidase (HRP) (Bio-Rad Laboratories; Hercules Calif.) for one hour, and then washed with TBST five times. RhoA bands were visualized on x-ray film by incubation of membrane with chemiluminescence luminol reagent (Santa Cruz Biotechnology) at room temperature for one minute.

After co-incubation for 2.5 days, lovastatin significantly inhibited the geranylgeranylation of RhoA at concentrations as low as 0.5 μM. The nonprenylated RhoA protein migrates more slowly than prenylated RhoA protein (McGuire et al., Oncogene 14:305-312 (1997)). Therefore, the increasing ratio observed between the higher apparent molecular weight RhoA protein and the lower apparent molecular weight RhoA protein with increasing concentration of lovastatin reflects decreasing isoprenylation of RhoA. Without lovastatin, the majority of RhoA in cells was isoprenylated. Most RhoA was in non-isoprenylated form with 14 μM lovastatin, which correlates with the inhibition of stimulated leukocyte adherence to FN. Addition of GGOH (5 μM) with lovastatin resulted in the more rapid migration of RhoA as seen in untreated cells. These results indicate that geranylgeranylation of RhoA protein is required for PMA-induced Jurkat cell adhesion.

These results show that lovastatin inhibits the geranylgeranylation of RhoA.

### EXAMPLE IV

### Inhibition of Lung Infiltration of Neutrophils by Lovastatin

This example demonstrates the inhibition of neutrophil emigration into lung of lipopolysaccharide-challenged mice by lovastatin.

In preliminary experiments, the distribution of an intra-nasal solution within the lungs of mice was determined. All protocols involving animals were reviewed and approved by the University of Washington animal Care and Use Committee and all experiments conform with the NIH guidelines of care and use of laboratory animals. To assess lung distribution following i.n. administration, sterile-endotoxin free carbon black was instilled into mice lungs to determine where the fluid was deposited. The carbon black was diluted in PBS, then an intra-nasal (i.n.) instillation of 2.5 μl/g (2.5 μl carbon black solution per g of mouse body weight) was completed. On a macroscopic level, the distribution of carbon black particles was relatively uniform throughout both lungs and within the lung. When examined at higher resolution, the distribution was somewhat patchy but penetrated to the alveoli within both lungs.

Pulmonary inflammation was induced in mice using a technique similar to that reported by others (Szarka et al., J. Immunol. Methods 202:49-57 (1997)). Briefly, mice were anesthetized with halothane, then 2.5 μl/g was placed into their nares where it was rapidly taken into the trachea and deep into the lungs. The solution instilled into the lungs contained lipopolysaccharide (LPS) (0.2 μg/μl; dissolved in PBS) (Sigma) and either lovastatin (0.28 μg/μl, 0.56 μg/μl or 2.8 μg/μl) or an equivalent amount of the carrier (˜2% ethanol). Compounds were administered in a given amount per g of mouse body weight. In a second set of experiments, PMA (50 ng/μl final concentration; 5 mg/ml stock solution in dimethyl sulfoxide) was used in place of LPS. Mice were allowed to recover from anesthesia and returned to their cages with free access to food and water.

At either 24 (LPS and PMA) or 48 (LPS) hours, mice were returned to the laboratory and killed, their chest rapidly opened, then both lungs were lavaged with 0.5 ml of normal saline followed by a second lavage of another 0.5 ml of normal saline that was lavaged in and out four times. The lavage fluid was combined, total cell counts determined using a hemocytometer, cytospin preparations were then made, the cells stained using the commercial kit (DiffQuik; Baxter Healthcare Corp.; Miami Fla.), and differential cells counts completed. The total number of mononuclear cells and neutrophils in the lavage fluid was determined by counting 100 cells. The number of neutrophils or macrophages was determined by multiplying the percentages by the total cell count. Differences between groups were tested using the computer program Stat View (Abacus Concepts; Berkeley Calif.) by analysis of variances and post hoc testing using Fisher's protected least square differences. Differences were assumed significant for p<0.05. The lavage fluid remaining after cytospin and cell counts was subjected to centrifugation, and the supernatant collected and frozen at −20° C. for later cytokine analysis. Based on the studies using carbon black, lavage of the lungs following instillation of LPS alone or LPS plus lovastatin results in a sample that is essentially an average of all of the lung. Thus, lavage fluid was derived from regions where LPS was deposited as well as those regions receiving no challenge.

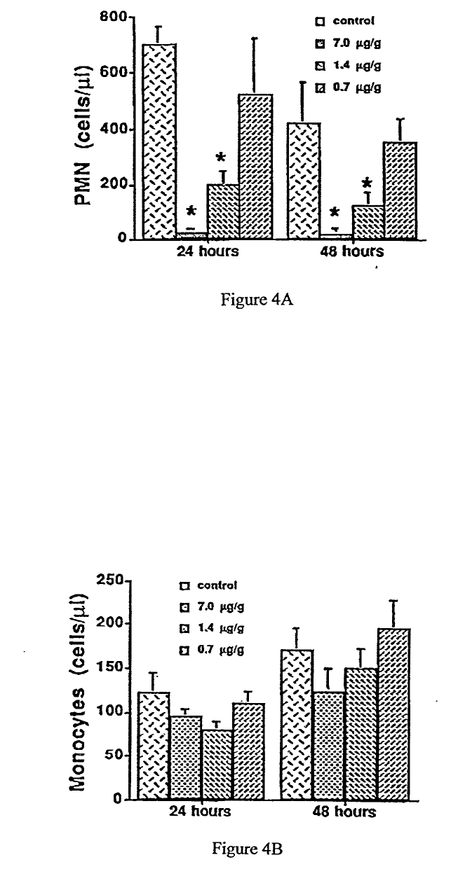

LPS stimulation in control animals for the i.n. lovastatin instillation had approximately 7.5×10⁵ PMNs/ml of lavage fluid 24 hours after LPS instillation (FIG. 4A). Simultaneous instillation of lovastatin with LPS resulted in a dose-dependent reduction in the number of PMNs in the lavage fluid at 24 hours. The control group (no lovastatin) was significantly different from the lovastatin groups given either 7.0 or 1.4 μg/g but not for the 0.7 μg/g group (p<0.05). The two groups receiving the higher doses of lovastatin were not different from each other. The percent of PMNs in the group given 7.0 μg/g, 1.4 μg/g and 0.7 μg/g of lovastatin compared with the control group was 4%, 28%, and 74%, respectively. The reduction in PMNs in the lavage fluid from lovastatin-treated mice killed at 48 hours was similar to that seen at 24 hours (FIG. 4A). The number of PMNs in the lavage fluid from the 48 hour control group was statistically different from the groups receiving either 7.0 μg/g or 1.4 μg/g of lovastatin (p<0.05); however, there was no difference between the control group and the group treated with the low dose lovastatin. The groups receiving either 7.0 μg/g or 1.4 μg/g of lovastatin were different from the low-dose group (p<0.05), but were not different from each other. At 48 hours, the percent of PMNs in the group given 7.0 μg/g, 1.4 μg/g and 0.7 μg/g of lovastatin compared with the control group was 5%, 32%, and 75%, respectively.

The number of mononuclear leukocytes in the lavage fluid from normal mice is approximately 1.0×10⁵ cells/ml. Monocyte emigration from blood into the alveolar space at 24 hours after LPS instillation was minimal (FIG. 4B), and lovastatin had no effect on the total number of lavage mononuclear leukocytes. The number of mononuclear leukocytes in lavage fluid increased to approximately 2.0×10⁵ by 48 hours after LPS instillation (FIG. 4B), and this number was somewhat less in those mice treated with lovastatin, although the differences were not statistically significant.

The method of administration of lovastatin was investigated in separate experiments. Mice were treated with lovastatin by intra-peritoneal (i.p.) injection 3 times at 24 hour intervals. The LPS was given at the same time as the last dose of lovastatin by i.n. instillation as described above. Treatment was with the vehicle for lovastatin or daily doses of 7.0 μg/g of lovastatin. Mice were returned to their cages with free access to food and water for 24 hours, then returned to the laboratory and killed. Lavage was performed and cell counts completed as described above.

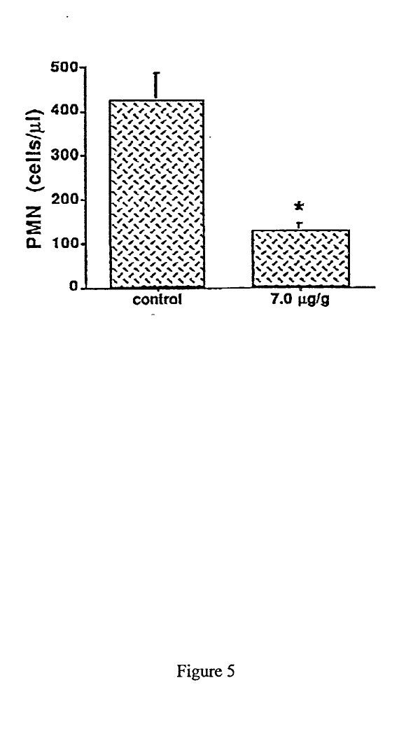

PMN emigration into the lung, when evaluated 24 hours after i.n. LPS and pretreatment with i.p. lovastatin, was significantly less (p<0.05) than in the control group (FIG. 5), as was observed with lovastatin administered i.n. There was only one treatment dose (7.0× μg/g) in the groups given i.p. lovastatin, and the number of PMNs accumulating in the lovastatin group was 30% of that in the control group. The number of mononuclear leukocytes in these two groups were not different between groups and was similar to that seen following treatment with i.n. lovastatin.

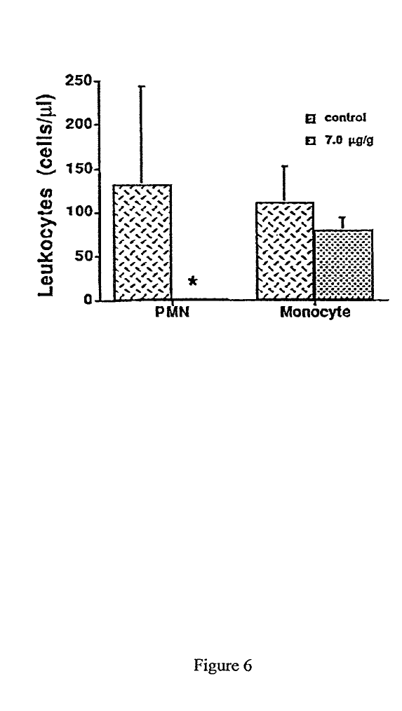

PMA given by i.n. instillation at the dose used in these experiments resulted in a modest infiltration of PMNs in alveolar lavage fluid at 24 hours, and essentially no change in alveolar mononuclear leukocytes. The simultaneous administration of lovastatin (7.0 μg/g) with PMA profoundly reduced the PMN emigration, resulting in essentially 100% inhibition (FIG. 6). There was no difference in mononuclear cell number between control and lovastatin treatment.

These results show that lovastatin inhibits the infiltration of neutrophils into lung in a mouse model of pulmonary inflammation.

### EXAMPLE V

### Inhibition of Cytokine Production by Lovastatin

This example demonstrates that lovastatin inhibits the production of cytokines in a mouse model of pulmonary inflammation.

Lavage fluids were used to measure cytokine production. The murine chemoattractant KC is a member of the CXC chemokine family of inflammatory cytokines and was measured by ELISA (R&D Systems; Minneapolis Minn.). The pro-inflammatory cytokine murine TNFα was also measured by ELISA (R&D Systems). The protocol for both assays was similar. Briefly, assay diluent was mixed with sample, which was the lavage fluid obtained as described in Example IV. The diluent was added to the appropriate capture plate and incubated for two hours. Plates were washed, incubated with conjugated anti-KC or anti-TNFα antibody for two hours, washed again, and then incubated with substrate for 30 min. A stop solution was then added, and the optical density determined by plate reader. Recombinant cytokine concentrations were assayed on each plate, and the test sample concentrations were determined from standard curves generated from these data.

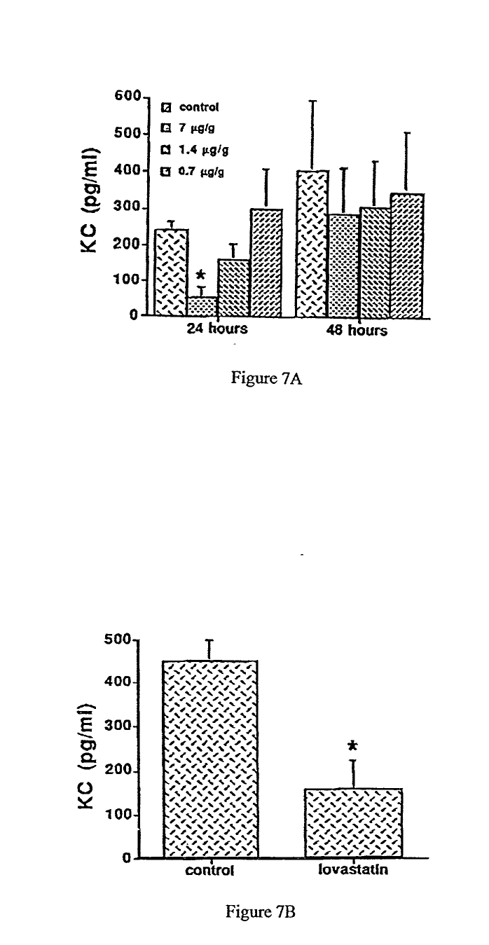

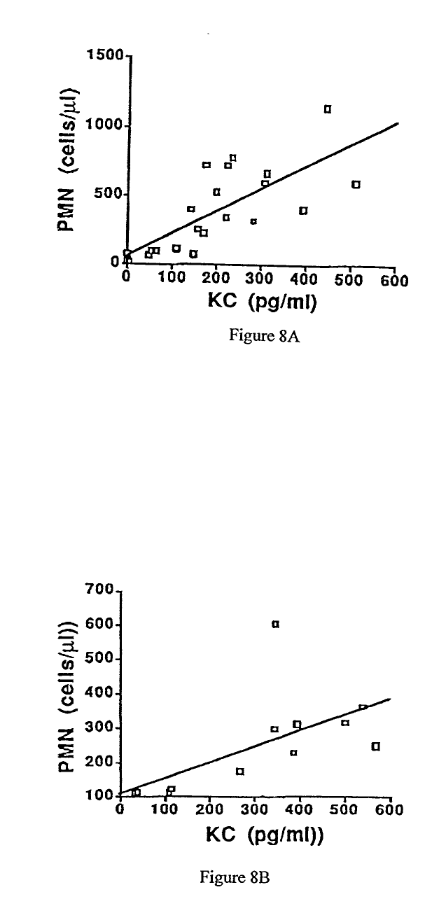

The concentration of KC chemokine in the lavage fluid of mice treated with i.n. lovastatin was reduced at 24 hours after instillation of LPS (FIG. 7A), and the group receiving 7.0 μg/g of lovastatin was significantly different from control. At 48 hours, the KC concentration was not different between the four groups of animals (FIG. 7A). Pretreatment with i.p. lovastatin also resulted in a significant reduction in KC concentration (FIG. 7B). PMN number versus KC concentration is shown for i.n. and i.p. lovastatin in FIG. 8A and 8B, respectively, for values determined at 24 hours. The line shows a linear regression best-fit for each of these groups and indicates increasing PMNs with increasing KC concentration. The correlation of PMNs with KC concentration was significant at 24 hours. At 48 hours, the correlation between PMNs and KC concentration was not significant.

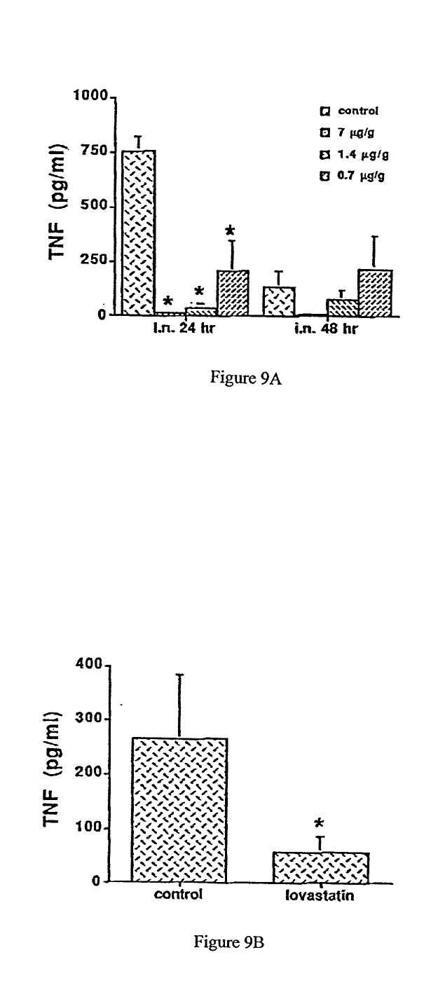

TNF concentration following i.n. treatment with lovastatin was reduced in a dose-dependent manner at 24 and 48 hours and differences between control and all lovastatin treatment doses were significant at 24 hours (FIG. 9A). Differences were not significant at 48 hours (FIG. 9A). Lovastatin given i.p. was also effective in preventing TNF production and these differences were statistically significant (FIG. 9B).

These results show that lovastatin inhibits cytokine production in a pulmonary inflammatory response in mice.

### EXAMPLE VI

### Inhibition of Leukocyte Infiltration into Lung by Lovastatin

This example demonstrates that lovastatin inhibits infiltration of leukocytes into lung while having no effect on airway responsiveness in a mouse model of asthma.

Asthma is characterized by airway hyperreactivity, has been attributed to chronic airway inflammation and is correlated with infiltration of eosinophils and lymphocytes into the airways. As a model of asthma, mice were sensitized with a mixture of ovalbumin and aluminum potassium sulfate (Henderson et al., J. Clin. Invest. 100:3083-3092 (1997)). Mice were given ovalbumin complexed with alum on days 0 and 14 by i.p. injection and further sensitized by i.n. instillation of ovalbumin on days 14, 25, 26 and 27. On day 28, these mice were challenged with methacholine and airway reactivity measured. This sensitization protocol results in mice with hyperreactive airways. Mice were treated with either lovastatin (7 μ/g) or vehicle, 2% ethanol in PBS, on days 25, 26, and 27 by combining the treatment with ovalbumin. Following measurement of airway reactivity, the mice were sacrificed, their lungs lavaged, and total and differential cell counts determined.

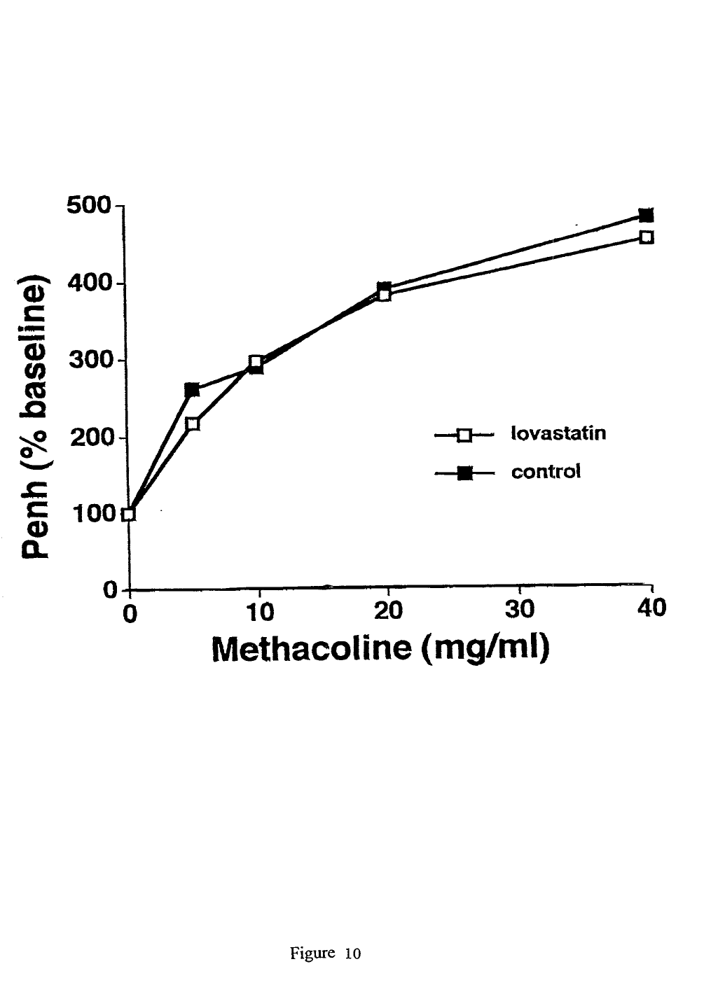

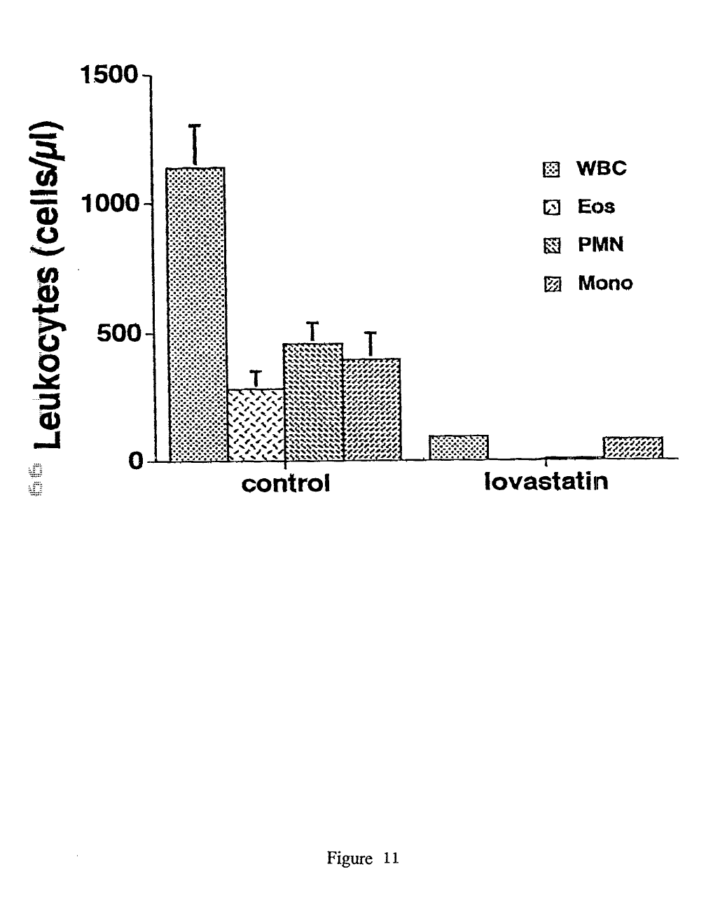

The airway reactivity, measured as PenH using a computerized plethysmograph (Buxco Electronics; Sharon Conn.), of the challenged mice was similar regardless of treatments (FIG. 10). However, treatment with lovastatin essentially blocked all leukocyte emigration into the lungs (FIG. 11).

These results show that lovastatin is effective in preventing leukocyte emigration into lung in an asthma model. The inhibition of leukocyte emigration can be useful in treating patients with asthma.

### EXAMPLE VII

### Anti-inflammatory Activity of Lovastatin Following Ischemia and Reperfusion

This example demonstrates that administration of lovastatin decreases inflammation following ischemia and reperfusion.

The effect of lovastatin on ischemia and reperfusion of the mouse hind limb was examined. Briefly, mice were anesthetized with halothane. A tourniquet was then placed on one hind limb to completely occlude blood flow and kept in place for 30 min. Body temperature was maintained at 37° C. with a heating blanket during the period of ischemia. Perfusion of the hind limb was re-established by removal of the tourniquet, and leg diameter was measured hourly for the next 6 hours and again at 24 hours. Mice (9 each for control and lovastatin treatment) were treated with either lovastatin or its carrier, 2% ethanol in PBS, by i.p. injection at the beginning of the ischemia. The lovastatin was given in a dose of 7 μg/g, and the carrier was given in an equal volume.

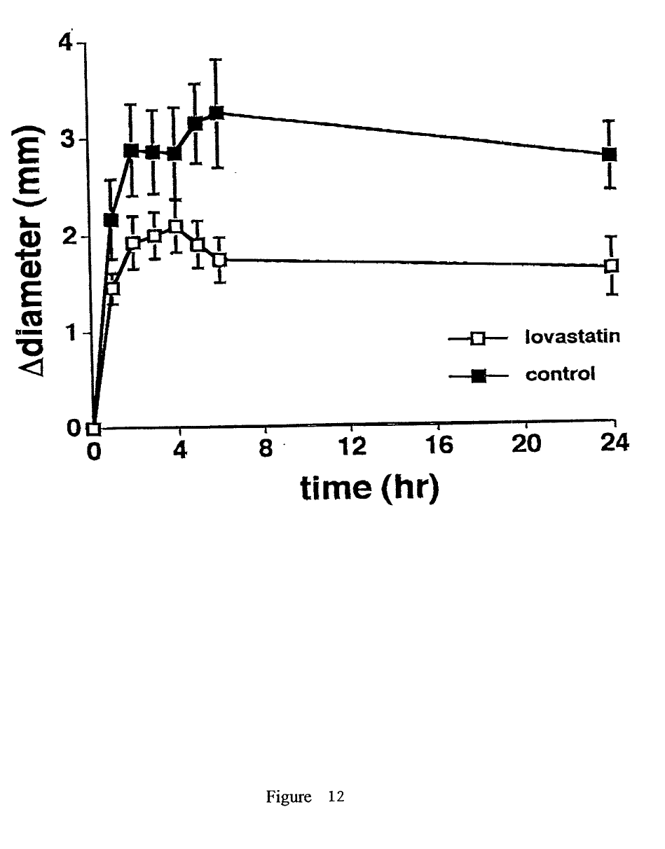

The above-described ischemia protocol resulted in increased leg diameter starting at the first measurement time and was still elevated at the 24 hour time point (FIG. 12). The vehicle treated leg had a significantly larger diameter than the lovastatin treated leg at 24 hours when compared by t-test.

These results show that lovastatin has anti-inflammatory activity. Due to the short time period of the experiment (24 hours), the anti-inflammatory activity is likely associated with an activity of lovastatin that is not directly associated with its ability to lower cholesterol.

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention. Accordingly, the invention is limited only by the claims.

What is claimed is:

1. A composition of matter comprising an aerosol formulation of a 3-hydroxy-3-methyl-glutaryl coenzyme A (HMG-CoA) reductase inhibitor, said HMG-CoA reductase inhibitor being present at a concentration of less than 0.1 mg.

2. The composition of claim 1, wherein said HMG-CoA reductase inhibitor is a statin.

3. The composition of claim 2, wherein said statin is lovastatin.

4. The composition of claim 2, wherein said statin is selected from the group consisting of pravastatin, simvastatin, cerivastatin, fluvastatin, atorvastatin and mevastatin.

5. The composition of claim 1, wherein said aerosol formulation comprises a propellant selected from the group consisting of halocarbons, hydrocarbons and ethers.

6. A method of treating a disease, comprising administering a pharmaceutical composition comprising an aerosol formulation of a HMG-CoA reductase inhibitor, wherein said disease is selected from the group consisting of acute lung injury, adult respiratory distress syndrome, asthma, interstitial lung disease, emphysema, chronic bronchitis and cystic fibrosis.

7. The method of claim 6, wherein said HMG-CoA reductase inhibitor is a statin.

8. The method of claim 7, wherein said statin is selected from the group consisting of lovastatin, pravastatin, simvastatin, cerivastatin, fluvastatin, atorvastatin and mevastatin.

9. The method of claim 6, wherein said HMG-CoA reductase is administered at less than 0.1 mg per day.

10. The method of claim 6, wherein said aerosol formulation comprises a propellant selected from the group consisting of halocarbons, hydrocarbons and ethers.

11. A method of treating acute lung injury, comprising administering a pharmaceutical composition comprising an aerosol formulation of a HMG-CoA reductase inhibitor.

12. The method of claim 11, wherein said HMG-CoA reductase inhibitor is a statin.

13. The method of claim 12, wherein said statin is selected from the group consisting of lovastatin, pravastatin, simvastatin, cerivastatin, fluvastatin, atorvastatin and mevastatin.

14. The method of claim 11, wherein said HMG-CoA reductase is administered at less than 0.1 mg per day.

15. The method of claim 11, wherein said aerosol formulation comprises a propellant selected from the group consisting of halocarbons, hydrocarbons and ethers.

16. A method of treating adult respiratory distress syndrome, comprising administering a pharmaceutical composition comprising an aerosol formulation of a HMG-CoA reductase inhibitor.

17. The method of claim 16, wherein said HMG-CoA reductase inhibitor is a statin.

18. The method of claim 17, wherein said statin is selected from the group consisting of lovastatin, pravastatin, simvastatin, cerivastatin, fluvastatin, atorvastatin and mevastatin.

19. The method of claim 16, wherein said HMG-CoA reductase is administered at less than 0.1 mg per day.

20. The method of claim 16, wherein said aerosol formulation comprises a propellant selected from the group consisting of halocarbons, hydrocarbons and ethers.

21. A method of treating asthma, comprising administering a pharmaceutical composition comprising an aerosol formulation of a HMG-CoA reductase inhibitor.

22. The method of claim 21, wherein said HMG-CoA reductase inhibitor is a statin.

23. The method of claim 22, wherein said statin is selected from the group consisting of lovastatin, pravastatin, simvastatin, cerivastatin, fluvastatin, atorvastatin and mevastatin.

24. The method of claim 21, wherein said HMG-CoA reductase is administered at less than 0.1 mg per day.

25. The method of claim 21, wherein said aerosol formulation comprises a propellant selected from the group consisting of halocarbons, hydrocarbons and ethers.

26. A method of treating emphysema, comprising administering a pharmaceutical composition comprising an aerosol formulation of a HMG-CoA reductase inhibitor.

27. The method of claim 26, wherein said HMG-CoA reductase inhibitor is a statin.

28. The method of claim 27, wherein said statin is selected from the group consisting of lovastatin, pravastatin, simvastatin, cerivastatin, fluvastatin, atorvastatin and mevastatin.

29. The method of claim 26, wherein said HMG-CoA reductase is administered at less than 0.1 mg per day.

30. The method of claim 26, wherein said aerosol formulation comprises a propellant selected from the group consisting of halocarbons, hydrocarbons and ethers.

31. A method of treating chronic bronchitis, comprising administering a pharmaceutical composition comprising an aerosol formulation of a HMG-CoA reductase inhibitor.

32. The method of claim 31, wherein said HMG-CoA reductase inhibitor is a statin.

33. The method of claim 32, wherein said statin is selected from the group consisting of lovastatin, pravastatin, simvastatin, cerivastatin, fluvastatin, atorvastatin and mevastatin.

34. The method of claim 31, wherein said HMG-CoA reductase is administered at less than 0.1 mg per day.

35. The method of claim 31, wherein said aerosol formulation comprises a propellant selected from the group consisting of halocarbons, hydrocarbons and ethers.

36. A method of treating cystic fibrosis, comprising administering a pharmaceutical composition comprising an aerosol formulation of a HMG-CoA reductase inhibitor.

37. The method of claim 36, wherein said HMG-CoA reductase inhibitor is a statin.

38. The method of claim 37, wherein said statin is selected from the group consisting of lovastatin, pravastatin, simvastatin, cerivastatin, fluvastatin, atorvastatin and mevastatin.

39. The method of claim 36, wherein said HMG-CoA reductase is administered at less than 0.1 mg per day.

40. The method of claim 36, wherein said aerosol formulation comprises a propellant selected from the group consisting of halocarbons, hydrocarbons and ethers.

41. A method of treating interstitial lung disease, comprising administering a pharmaceutical composition comprising an aerosol formulation of a HMG-CoA reductase inhibitor.

42. The method of claim 41, wherein said interstitial lung disease is selected from the group consisting of interstitial pneumonitis, interstitial fibrosis, and idiopathic fibrosis.

43. The method of claim 41, wherein said HMG-CoA reductase inhibitor is a statin.

44. The method of claim 43, wherein said statin is selected from the group consisting of lovastatin, pravastatin, simvastatin, cerivastatin, fluvastatin, atorvastatin and mevastatin.

45. The method of claim 41, wherein said HMG-CoA reductase is administered at less than 0.1 mg per day.

46. The method of claim 41, wherein said aerosol formulation comprises a propellant selected from the group consisting of halocarbons, hydrocarbons and ethers.
